(19) United States
(12) Patent Application Publication
(10) Pub. No.: US 2024/0194288 A1
(43) Pub. Date: Jun. 13, 2024

(54) GENERALIZED NESTEDNESS DETECTION IN MICROBIAL COMMUNITIES

(71) Applicant: International Business Machines Corporation, Armonk, NY (US)

(72) Inventors: Niina Haiminen, Tampere (FI); Laxmi Parida, Mohegan Lake, NY (US); Filippo Utro, Pleasantville, NY (US)

(21) Appl. No.: 18/079,186

(22) Filed: Dec. 12, 2022

(51) Int. Cl. G16B 5/00; C12Q 1/04; G16B 45/00

(52) U.S. Cl. CPC G16B 5/00; C12Q 1/04; G16B 45/00

(57) ABSTRACT

Topological data analysis is used to detect general nestedness in microbial communities through the generation and application of filtration matrices and persistent homology barcodes. From a starting point of an input matrix with 1s and 0s, a filtration matrix is generated with a mathematical function, such as a Jaccard similarity, overlap coefficient, or a min(com1/sum(1)) computation. The persistent homology of the filtration matrix is then calculated in at least two dimensions using a mathematical function, such as a simplicial complex, a cubical complex, an alpha complex, a C̆ech complex, or a Vietris-Rips complex, that is displayed in a barcode that provides a visualization of the nestedness within the microbial community. P-values for the microbial community nestedness can be calculated by comparing the shape and length of the persistent homology barcodes for the input matrix against persistent homology barcodes for a completely randomized input matrix.

### TECHNICAL FIELD

The present invention relates generally to nestedness in microbial communities, and more specifically to general nestedness detection in microbial communities through topological data analysis.

### BACKGROUND OF THE INVENTION

Nestedness explains the structure of metacommunities in which species-poor sites present as subsets of the biota of species-rich sites. In mutualistic, parasitic, and trophic networks, nestedness reveals that the interactions of less connected specialist species are often subsets of the interactions of highly connected generalist species. Within generalized nestedness, specialist species form distinct nested patterns.

The nestedness of microbial communities provides information on: (i) climate, geography, and built environment-related patterns across the earth microbiome; (ii) human microbiome in different individuals; (iii) human microbiome across niches in an individual; and (iv) phytobiomes across a plant or plants. Quantifying the nestedness of generalist species within microbial communities increases the understanding of community ecology and the health or illness of an environment or individuals within that environment. In order to obtain such information, the nested microbe species within the microbial community require quantification. Presently, means for such quantification remain a need in the art.

### SUMMARY OF THE INVENTION

The present invention overcomes the need in the art by providing a method of de-noising and quantifying microbial community data through nested topological data analysis (nesTDA) of original microbial input data. From a starting point of an original input matrix for samples taken from the microbial community, the nesTDA includes the generation of filtration matrices and persistent homology barcodes for the original input data matrix. The shape and length of the persistent homology barcodes are analyzed in at least one dimension in order to assess the degree of nestedness of the microbes in the microbial community.

In one embodiment, the present invention relates to a computer-implemented method for analyzing nestedness of microbes in a microbial community comprising: generating an input matrix with microbe data comprising 1s and 0s for samples taken from a microbial community, wherein 1 represents the presence of a microbe in the samples and 0 represents the absence of microbes in the samples; generating a filtration matrix that identifies similarities between microbes in the samples; and generating at least one persistent homology barcode for the filtration matrix, wherein the at least one persistent homology barcode has a shape and length that provides information on the nestedness of the microbes in the microbial community.

In another embodiment, the present invention relates to a computer-implemented method for analyzing nestedness in a microbial community comprising: generating an input matrix with 1s and 0s for one or more samples from the microbial community, wherein 1 represents the presence of a microbe in the one or more samples and 0 represents the absence of microbes in the one or more samples; computing a filtration matrix for the one or more samples; computing a C̆ech complex persistent homology for the filtration matrix in at least two dimensions; and displaying the persistent homology of the filtration matrix with at least one persistent homology barcode, wherein the at least one persistent homology barcode has a shape and length that provides information on the nestedness of the microbes in the microbial community.

In a further embodiment, the present invention relates to a computer-implemented method for analyzing nestedness in a microbial community comprising: generating an input matrix with 1s and 0s for one or more samples from the microbial community, wherein 1 represents the presence of a microbe in the one or more samples and 0 represents the absence of microbes in the one or more samples; computing a Jaccard similarity filtration matrix for the one or more samples; computing a C̆ech complex persistent homology for the filtration matrix in at least two dimensions; and displaying the persistent homology of the filtration matrix with at least one persistent homology barcode, wherein the at least one persistent homology barcode has a shape and length that provides information on the nestedness of the microbes in the microbial community.

In another embodiment, the present invention relates to a computer-implemented method for analyzing nestedness in a microbial community comprising: generating an input matrix with 1s and 0s for one or more samples from the microbial community, wherein 1 represents the presence of a microbe in the one or more samples and 0 represents the absence of microbes in the one or more samples; computing a filtration matrix with a min(com1/sum(1)) computation for the one or more samples; computing a C̆ech complex persistent homology for the filtration matrix in at least two dimensions; and displaying the persistent homology of the filtration matrix with at least one persistent homology barcode, wherein the at least one persistent homology barcode has a shape and length that provides information on the nestedness of the microbes in the microbial community.

In a further embodiment, the present invention relates to a computer program product for analyzing nestedness in a microbial community comprising: program instructions on one or more computer readable storage media for generating an input matrix with 1s and 0s for one or more samples from the microbial community, wherein 1 represents the presence of a microbe in the one or more samples and 0 represents the absence of microbes in the one or more samples; program instructions on one or more computer readable storage media for computing a filtration matrix for the one or more samples; program instructions on one or more computer readable storage media for computing persistent homology for the filtration matrix in at least two dimensions; and program instructions on one or more computer readable storage media for displaying the persistent homology of the filtration matrix with at least one persistent homology barcode, wherein the at least one persistent homology barcode has a shape and length that provides information on the nestedness of the microbes in the microbial community.

In another embodiment, the number of 1s in the matrix also represents abundance of microbes in the samples.

In a further embodiment, the filtration matrix is generated with a mathematical function selected from the group consisting of Jaccard similarity, overlap coefficient, or a min(com1/sum(1)) computation.

In another embodiment, the persistent homology is generated with a mathematical function selected from the group consisting of simplicial complex, a cubical complex, an alpha complex, a C̆ech complex, or a Vietris-Rips complex.

In a further embodiment, a p-value of the nestedness in the microbial community is calculated by comparing the barcodes generated from the input matrix against barcodes generated from the input matrix with randomly permutated microbe data.

In another embodiment, the microbe data is randomly permutated by introducing noise into the samples, wherein the input matrix and the randomly permutated input matrix have the same number of 1s and 0s.

Additional aspects and/or embodiments of the invention will be provided, without limitation, in the detailed description of the invention that is set forth below.

### BRIEF DESCRIPTION OF THE DRAWINGS

FIG. 1A is a matrix showing single nestedness at sampled sites and FIG. 1B is a matrix depicting generalized nestedness at sampled sites.

FIG. 2A shows two matrices, one with 0% noise in the input data and the other with 20% noise added to the input data; FIG. 2B shows two matrices with 40% and 60% noise added to the input data; and FIG. 2C shows two matrices with 80% and 100% noise added to the input data.

FIG. 3A shows an input data matrix with Jaccard similarity and min(com1/sum(1)) barcodes at H0 and H1 dimensions for microbes in a sample with nested observed data; FIG. 3B shows an input data matrix with Jaccard similarity and min(com1/sum(1)) barcodes at H0 and H1 dimensions for microbes in a sample with 30% noise; and FIG. 3C shows an input data matrix with Jaccard similarity and min(com1/sum(1)) barcodes at H0 and H1 dimensions for microbes in a randomly permutated sample.

FIG. 4A shows an input matrix with Jaccard similarity barcodes at H0, H1, and H2 dimensions for microbes in a sample with nested observed data; and FIG. 4B shows an input matrix with Jaccard similarity barcodes at H0, H1, and H2 dimensions for microbes in a randomly permuted sample.

FIG. 5 is a schematic diagram of a computer environment that may be used to implement the nestedness detection methods described herein.

### DETAILED DESCRIPTION OF THE INVENTION

Set forth below is a description of what are currently believed to be preferred aspects and/or embodiments of the claimed invention. Any alternates or modifications in function, purpose, or structure are intended to be covered by the appended claims. As used in this specification and the appended claims, the singular forms “a,” “an,” and “the” include plural referents unless the context clearly dictates otherwise. The terms “comprise,” “comprised,” “comprises,” and/or “comprising,” as used in the specification and appended claims, specify the presence of the expressly recited components, elements, features, and/or steps, but do not preclude the presence or addition of one or more other components, elements, features, and/or steps.

As used herein, the term “microbial community” to refer to a group of microbes that share a common living space. The microbes in the community may be one or more microbe species, strains, genus, and/or taxonomic levels that share features in a sampled site, such as a physical location and/or a function. In the discussion that follows, where term “microbe species” is used, this term is used solely for purposes of illustration and is not meant to be limiting with respect to the larger scope of the microbes referenced herein.

As used herein, the term “matrix” and “matrices” refers to graphical representations of a network having rows, columns, and cells where each matrix cell is filled with 1 if the element (e.g., a microbe) represented in a column interacts or is related to an element in a row. If there is no interaction, the matrix cell is filled with 0. The matrices described herein include, without limitation, nestedness matrices that relate to the presence, absence, and abundance of all microbes in microbial community samples; randomized matrices that represent a randomization of a nestedness matrix (through the introduction of noise) for purposes of empirical comparison and p-value computation; and filtration matrices that are mathematically generated from nestedness and randomized matrices and which are described in detail below.

As used herein, the term “p-value” refers to a statistical analysis that produces test results that are an extreme of the original observed data. A p-value computation is used to test that a null hypothesis is correct. A null hypothesis posits that two possibilities are the same and that observed data is due to chance alone. A small p-value means that an extreme observed outcome is unlikely under the null hypothesis and a large p-value means that an extreme observed outcome is likely under the null hypothesis. Within the context of the present invention, the extreme of the original observed data is a completely randomized matrix of the data in the original data of observed data.

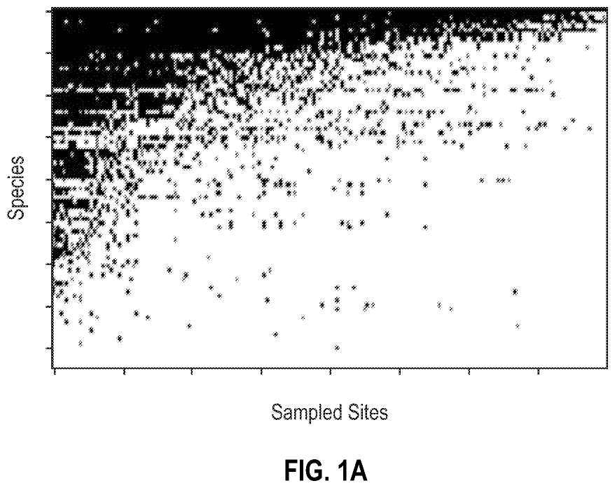

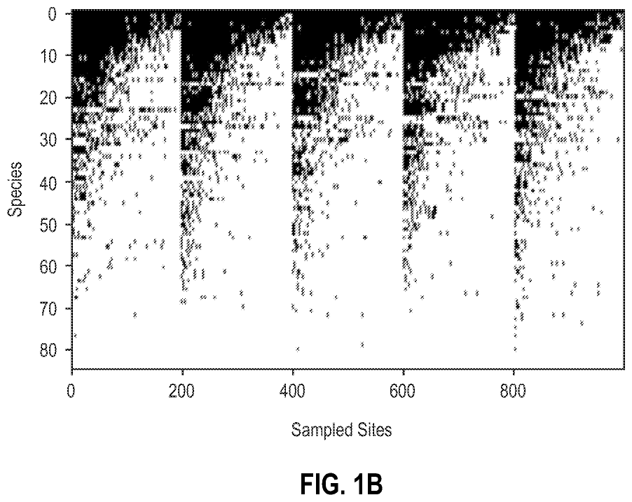

As used herein, the term “nestedness” refers to general nestedness in microbial communities where the interactions of less connected (specialist) microbes form subsets of the interactions of more connected (generalist) microbes. FIG. 1A is a nestedness matrix depicting single nestedness of microbe species at sampled sites within a microbial community and FIG. 1B is a nestedness matrix showing generalized nestedness of microbe species at sampled sites within a microbial community. As shown in FIG. 1B, in generalized nestedness, the subsets of species form distinct nested patterns, which must be quantified to recover the nested elements.

As used herein, the term “noise” refers to outliers in a nestedness matrix. In a perfectly nested matrix, a hypothetical boundary line separates the occupied area of the matrix from the unoccupied portion. Noise is a feature of imperfect matrices where elements (e.g., a microbe species) appear in the unoccupied area of the hypothetically perfect matrix.

As used herein, the term “nesTDA” refers to topological data analysis (TDA) that quantifies the nestedness of the microbes within a microbial community. TDA generally refers to a mathematical application that gives shape to data by combining algebraic topology and computational geometry to provide qualitative and quantitative information about the structure of data. The input for TDA is typically a matrix made up of a finite set of points that have distance or similarity between them. A continuous shape is built on top of the data in order to highlight the underlying topology or geometry. Topological or geometric information is extracted from the structures built on the top of the data resulting in a full reconstruction (e.g., a triangulation) of the shape underlying the data from which topological/geometric features can be extracted. The extracted topological and geometric information provides new families of features and descriptors of the data, which can be used to better understand the data through visualization.

Application of the nesTDA to microbial community data requires application of two sets of mathematical functions, application of a first mathematical function to establish a filtration matrix followed by application of a second mathematical function to connect the data within the filtration matrix. Since nesTDA is continuous function, the mathematical functions that generate the nesTDA consider the influence of noise on nested and unnested elements. Further, because nesTDA considers k-wise overlap (k<m, n) (rather than pair-wise overlap), nesTDA de-noises microbial community data by considering the effect of microbe species that appear below the hypothetical boundary of the data matrices. The input for nesTDA is an original data matrix of elements (also referred to herein as an “input matrix) and the output is a visualization of the nestedness elements within the input matrix.

As used herein, the term “filtration matrix” refers to a matrix that finds similarities between all elements in one or more samples. Within the context of the present invention, the filtration matrices find similarities between all microbe species within a microbial community through the application of mathematical functions, such as Jaccard similarity, overlap coefficient, or the minimum of the common microbe component over the total number of microbes in a sample, the latter of which is referred to herein as min(com1/sum(1)) and which may be defined according to the following metric:

$$min ( \frac{\vert A ⋂ B \vert}{\vert A \vert} , \frac{\vert A ⋂ B \vert}{\vert B \vert} )$$

As used herein, the term “Jaccard Similarity” refers to a graph application that measures the similarity between two sets based upon the intersection of the number of common elements between the two sets over the number of unique elements in each set. For two sets, A and B, the Jaccard Similarity is defined by the following metric:

$$J ( A , B ) = \frac{\vert A ⋂ B \vert}{\vert A ⋃ B \vert} = \frac{\vert A ⋂ B \vert}{\vert A \vert + \vert B \vert - \vert A ⋂ \vert B \vert} .$$

Applying this metric, 0≤J(A,B)≤1; thus, if the intersection between A and B, or the converse, is empty, then J(A,B)=0.

As used herein, the term “overlap coefficient” refers to a graph application that measures the size of the intersection of two sets over the size of the smaller set. For two sets, A and B, the overlap coefficient is defined by the following metric:

$$overlap ( A , B ) = \frac{\vert A ⋂ B \vert}{min ( \vert A \vert , \vert B \vert )} .$$

Applying this metric, where A is a subset of B, or the converse, the overlap coefficient is 1.

As used herein, the term “persistent homology” refers to a mathematical method of computing topological features of a space at different spatial resolutions. Where a greater number of persistent features are detected over a wide range of spatial scales, the high number of persistent features are deemed more likely to represent true features of the underlying space, rather than artifacts of sampling, noise, or a particular choice of parameters. Within the context of the present invention, persistent homology is applied to a microbial community in order to identify similarities of the microbes therein. To find the persistent homology of a microbial community, the community, as represented in a filtration matrix, is mathematically analyzed at different spatial resolutions, the latter of which are displayed as barcodes.

As used herein, the term “barcode” and “persistent homology barcode” refers to a graphical representation of the persistent homology of a dataset. A barcode is a graphical representation of the life span of connected points within a topological space as the relationship between the points increases through the application of a mathematical function to the data in the topological space. A persistence homology barcode appears as a collection of horizontal line segments in a plane whose horizontal axis corresponds to a parameter interval (e.g., time, distance between features, overlap between features) and whose vertical axis represents an arbitrary ordering of homology generators that come into existence at each parameter as a result of the increasing relationship between the points. The barcode thus represents a birth-death pairing of homology generators (with the exception of those that persistent to infinity) where the horizontal line segments correspond to the homology generators that appear at a first parameter and disappear at a second parameter (e.g., two different points in time, distance between two separate features, overlap between two separate features). Examples of mathematical functions that may be used to generate persistent homology barcodes include, without limitation, simplicial complexes, cubical complexes, alpha complex, C̆ech complexes (C̆ε(X)), or Vietris-Rips complexes.

Simplicial complexes are a set composed of points, lines, triangles, and their n-dimensional counterparts. A simplicial complex K is a set of simplices where (1) every face of a simplex form K is also in K and (2) the non-empty intersection of any two simplices S₁, S₂∈K is a face of both S₁ and S₁.

Cubical complexes are a set composed of points, line segments, squares, cubes, and their n-dimensional counterparts. A cubical complex K is a set of cubes of any dimension where (1) every face of a cube from K is also in K and (2) if two cubes in K have a non-empty intersection, then the intersection is a face of each simplex.

Alpha complexes are simplicial complexes constructed from the finite cells of a Delaunay Triangulation (DT), where for a given set of discrete points P, DT(P) has no point that is inside the circumcircle of any triangle. DTs maximize the minimum of all of the angles of the triangles in the triangulation and thus avoid sliver triangles (because for a set of points on the same line, there is no DT).

A Vietris-Rips complex is an abstract simplicial complex that is defined from any metric space M and distance δ by forming a simplex for every finite set of points that has a diameter that is at most δ. For a family of finite subsets of M in which a subset of points K form a K−1 dimensional simplex (the simplest possible polytope made with line segments in any direction e.g., an edge for two points, a triangle for three points, and a tetrahedron for four points, etc.), S is a simplex within a complex if the distance between every pair of points in in a finite set S is at most δ.

A C̆ech complexes is also an abstract simplicial complex, but it is constructed from a point cloud in a metric space, which has a simplex for every finite subset of balls with non-empty intersections. For a finite point cloud X and a number ε>0, the C̆ech complex C̆ε is the simplicial complex whose simplices are formed as follows: for each subset S⊏X of points, form a ball (ε/2) around each point in S and include S as a simplex if there is a common point contained in all of the balls in S. The C̆ε(X) is thus the nerve of the set of ε-balls centered at points of X.

Persistent homology barcodes can be generated for different dimensions within a topological space where the dimension is represented by a homology group, H0, H1, H2, where 0, 1, 2 are Betti numbers that classify topological spaces according to the increasing connectivity of segments within the space. Within a topological space represented by geometric shapes formed through the connection of discrete points, as the radii between the points increases, the intersections between the points also increases, thus forming geometric shapes with increasing complexity. The complexity of the geometric shapes within the topological space are characterized by the homology group dimensions, H0, H1, and H2 where H0 detects the connectedness of discrete points that have no boundary; H1 detects 1-dimensional holes in a complex with connected points that share a boundary; and H2 captures 2-dimensional voids within highly connected points that share at least one boundary. The connectivity of the points at the different dimensions is based upon the mathematical function that is used to generate the barcodes.

As noted above, a persistent homology barcode comprises horizontal segments that represents a birth-death pairing of homology generators, the latter of which comprises the increasing radii of the geometric shapes within the topological space. As the radii of the points varies, there is a complex in which it first appears (i.e., the complex is born) and a complex in which the cycle has been completed filled in or has become a boundary to a higher dimensional simplex (i.e., the complex dies). The lifetime of the cycle is the difference in time of death and time of birth. Cycles with long lifetimes are called persistent and are likely to give topological information about the entire data set. The barcode shows how much of the topology persists over a set parameter by visualizing all of the cycles that appear in a data set along with their lifetimes and when they appear in relation to all other cycles. Within a barcode, the x-axis is the radius value, r, and each cycle's bar starts at its birth time and ends at its death time; thus, persistent cycles are represented by long bars while short bars are considered insignificant due to noise in the data. Because real data is noisy, knowing what noise in a data set looks like in a persistent homology barcode helps to rule out the noise and identify the significant homological features in the data set. Within the context of the present invention, barcodes represent the persistent homology of nested microbes within a microbial community by visualizing the geometric interpretation of the nested microbes at different dimensions (e.g., H0, H1, H2) for the original observed data and if appropriate, data with added noise and/or completely permutated data.

The present invention provides a method of applying nesTDA to de-noise data and to quantify nestedness in microbial communities. In one embodiment, a method of analyzing nested patterns in a microbial sample comprises: (1) generating an input matrix that includes data relating to microbe presence, absence, and abundance in samples from a microbial community; (2) generating a filtration matrix for all of the microbes in the samples; (3) calculating the persistent homology within the filtration matrix and displaying the persistent homology with a barcode; and (4) analyzing the persistent homology barcode to determine a level of microbe nestedness within the microbial community.

In another embodiment, the filtration matrix is generated with a Jaccard similarity, overlap coefficient, or a min(com1/sum(1)) computation.

In a further embodiment, the persistent homology is calculated with a mathematical function selected from the group consisting of a simplicial complex, a cubical complex, an alpha complex, a C̆ech complex, or a Vietris-Rips complex. In another embodiment, the persistent homology is calculated in at least two dimensions. With reference to FIG. 1A, the nestedness shown therein is for a single dimension. By contrast, FIG. 1B shows nestedness in multiple dimensions. In a further embodiment, the persistent homology is displayed with barcodes. Regardless of the mathematical formula used to calculate the persistent homology and the associated dimension, a barcode with a smooth transition indicates nestedness of microbes in a microbial community and a stepwise transition indicates that there is no microbial nestedness in the microbial community.

In another embodiment, noise is added to an input matrix for the purpose of empirical comparison and p-value computation. When adding random noise to an input matrix, No, the number of 1s in the random noise matrix, Nr, must be the same as the number of 1s in the input matrix since changing the number of 1s from the No to the Nr can negatively impact the nesTDA of the data. In order to add noise to an input matrix, the following steps are taken:
1. Generate No for microbial community data with rows, columns, and cells, where the cells include 0s and 1s, where the 1s represent the presence of a microbe and the 0s represent the absence of a microbe;
2. Generate an empty matrix, M, with the same number of rows, columns, and cells as the No, but where all of the cells in the matrix are 0s;
3. Select a noise percentage, x %, of the number of 1s in the No, where x is a number between 0-100 such that 0% noise is the noise in the original matrix and 100% noise is the noise in a totally random matrix;
4. Assign No(x)=N*(100−x)/100 positions in M that were 1 in the original data set as 1, where N=the number of 1s in the No;
5. Assign Nr(x)=N*x/100 random positions in M as 1 among the entire matrix, where N=the number of 1s in the No;
6. If any position in step 5 overlaps with a position in step 4, the random assignments are repeated until there are no overlaps; and
7. Output new matrix M.

In a further embodiment, steps 5 & 6 above are altered such that an up-to-date list of empty positions in M is kept and those empty positions are used for the random 1 assignments in step 6. In practice, once the x % of the number of 1s in No is chosen, the 1s are placed in M in the same row and column as in No, but in a random position until there are no overlaps.

In another embodiment, p-values for the microbial community nestedness can be calculated by comparing the persistent homology barcode for the input matrix against a persistent homology barcode for a completely randomized input matrix.

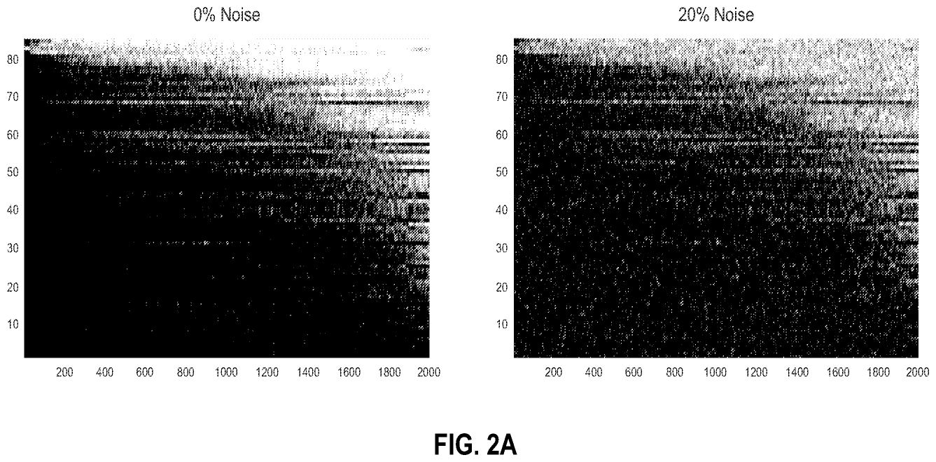

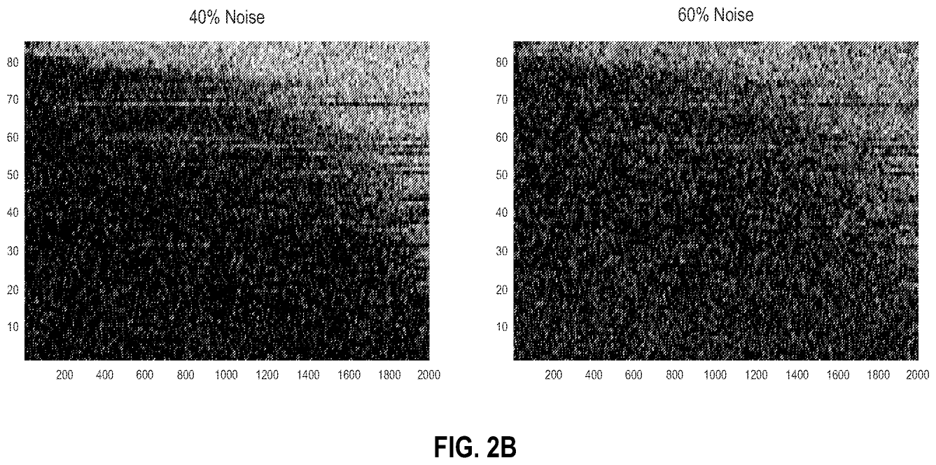

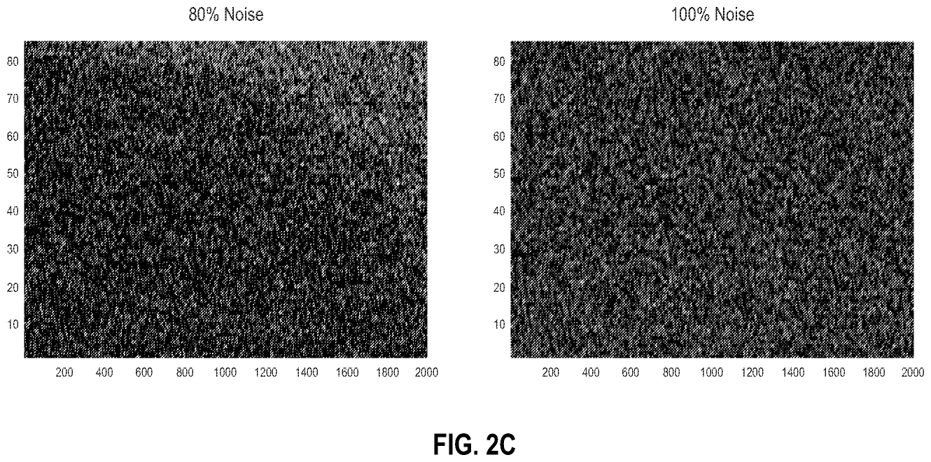

FIGS. 2A-2C shows six input matrices with varying amounts of noise added to original observed input data. FIG. 2A shows one input matrix of original observed nested data with 0% noise added and another input matrix with 20% noise added to the original data. FIG. 2B shows two input matrices, one with 40% noise added to the original data and the other with 60% noise added to the original data. FIG. 2C shows two input matrices, one with 80% noise added to the original data and the other with 100% noise added to the original data. The 100% noise results in a completely random matrix. An exemplary method for generating the noisy matrices of FIGS. 2A-2C is described in Example 1.

The introduction of noise into the input matrix changes the input matrix where all the is that occupy a space representing the original data are changed to a completely random matrix. In the random matrix, the number of 1s in the original matrix remain the same, but the placement of the 1s is different from the placement of the 1s in the original input matrix. The matrix with the random placement of 1s represents the original matrix with noise.

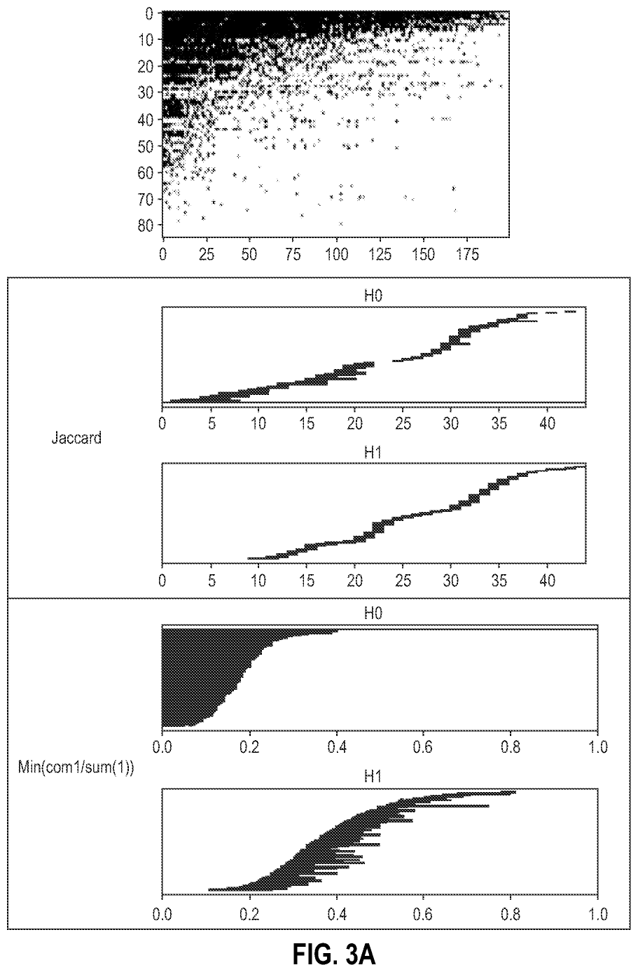

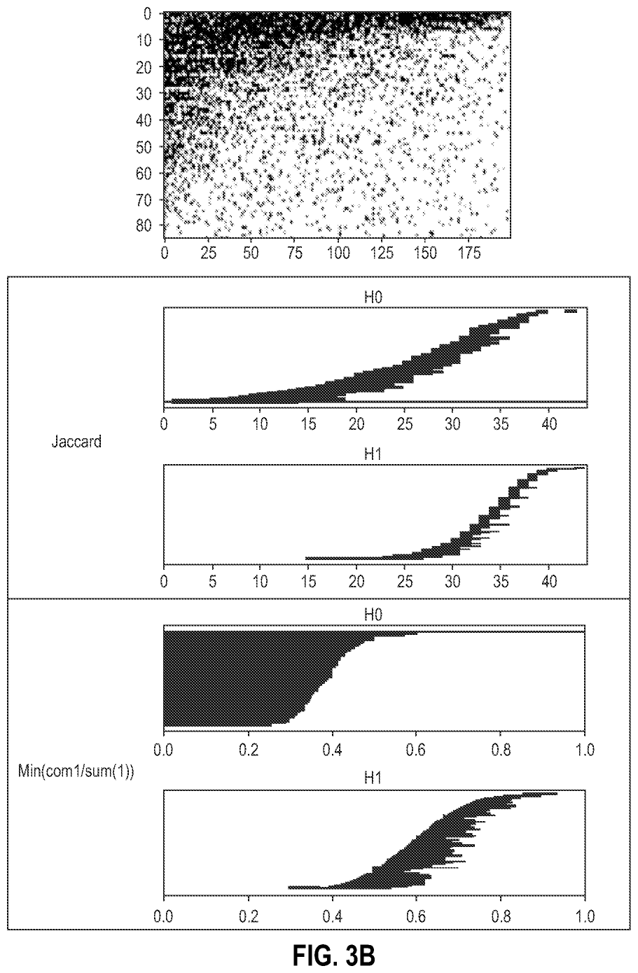

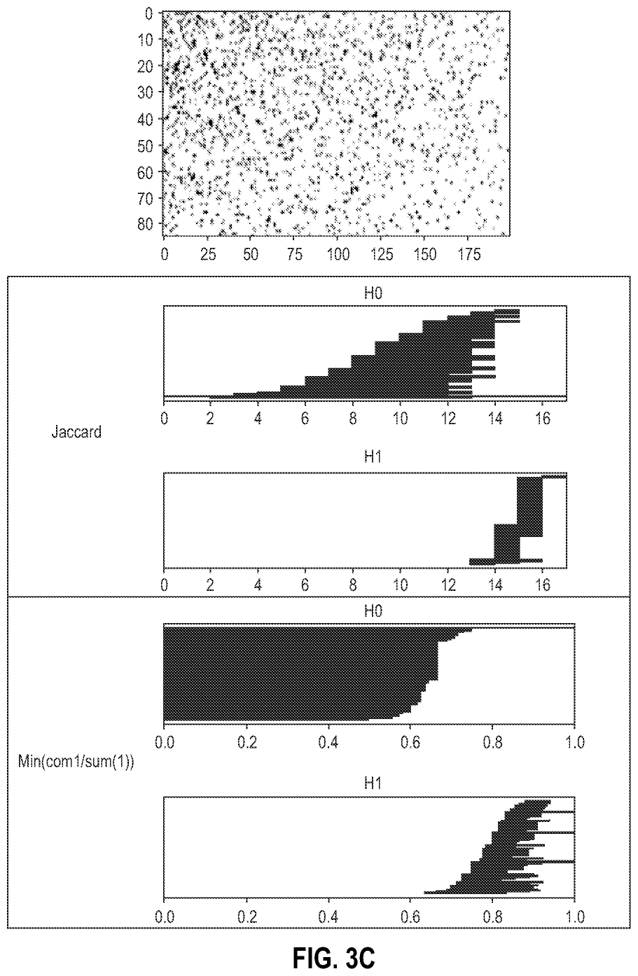

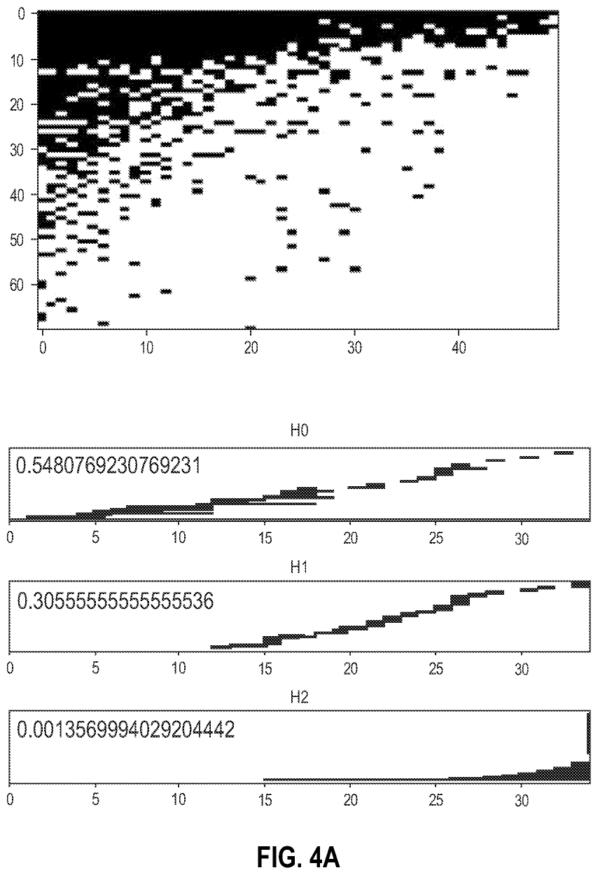

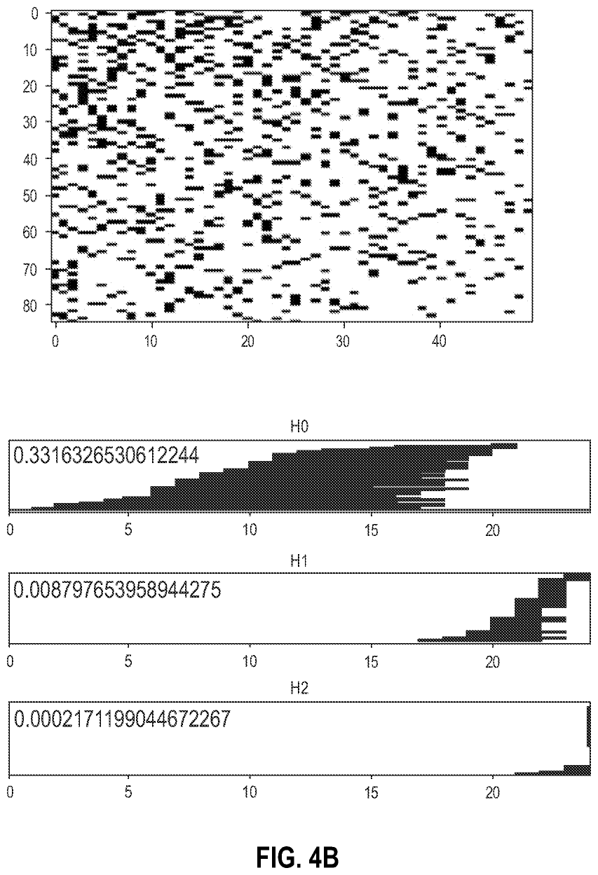

FIGS. 3A-3C and FIGS. 4A-4B show the application of the nesTDA method to samples from a microbial community in order to visualize the nesting of microbes in the microbial community. The nesTDA analysis includes analyzing the shape and length of the persistence homology barcodes shown therein. An exemplary method for generating the homology barcodes of FIGS. 3A-3C and FIGS. 4A-4B is described in Example 2.

FIG. 3A-3C shows an original observed nested input data matrix (FIG. 3A), an input matrix with 30% added noise (FIG. 3B), and an input matrix with completely permutated data (FIG. 3C), where rows correspond to microbes, columns correspond to samples, and the presence of a microbe in a sample is represented by a 1. Also shown are H0 and H1 persistent homology barcodes generated for each of the three matrices via C̆ech complex calculations on Jaccard similarity and min(com1/sum(1)) filtration matrices (referred to herein as “Jaccard barcodes” and “min(com1/sum(1)) barcodes”). In a perfectly nested matrix, the set of microbes in the samples and the sets of samples at which microbes are present form proper subsets of each other.

In FIGS. 3A-3C, the original observed nested input data matrix and its barcodes serve as a control for the 30% noise and randomly permuted data matrices. On the randomly permuted matrix, both of the H1 dimension barcodes appearing late in the filtration time frame (the x-axis), which indicate that there is no nested structure in the randomly permuted matrix at the H1 dimension. In other words, the randomly permuted matrix does not include general microbial nesting (as represented by FIG. 1B) and only includes a nesting of a single microbial species (as represented by FIG. 1A). This outcome is consistent with the nested observed data. The similarity of the barcode curves between the nested observed data and the nested with 30% noise indicates that the method described herein is able to predict nestedness in a microbial sample with noise as well as it is in a microbial sample that does not include noise.

FIGS. 4A-4B show the higher H2 dimension for an input matrix (FIG. 4A) and its randomly permutated counterpart matrix (FIG. 4B) for microbes in a microbial community as well as H0, H1, and H2 dimension barcodes generated with C̆ech complex calculations on Jaccard similarity filtration matrices. The numbers associated with each barcode represent the area under the curve (AUC), which is one of many methods known to those of skill in the art for measuring the two-dimensional area under a receiver operating characteristic (ROC) curve, such as the barcodes shown herein. H2 barcode provides additional information on the nesting of the microbes in the tested microbial community. The very small area under the curve for both the original and randomized data confirms that the microbes in the sample tested therein does not have generalized data. In samples where the H1 barcodes indicate nesting, then H2 or higher dimensions should be tested on microbial community samples to determine how many layers of microbe nesting exist in the microbial community.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random-access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

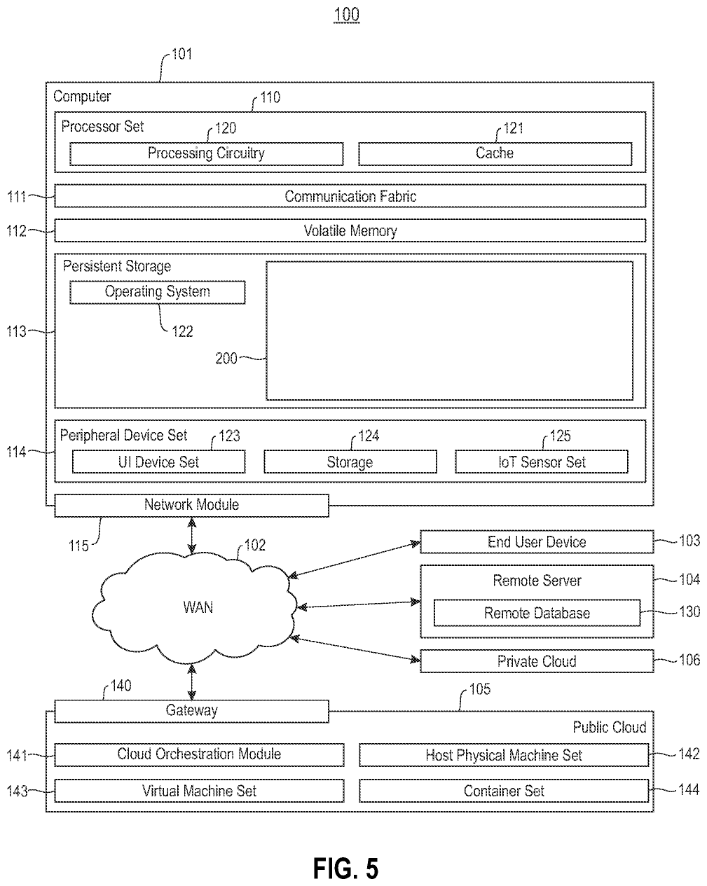

The following discussion refers to FIG. 5. Computing environment 100 contains an example of an environment for the execution of at least some of the computer code involved in performing the inventive methods, such as codes for generating input matrices, filtration matrices, and persistent homology barcodes for microbial community samples 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 5. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid-state storage devices. Operating system 122 may take several forms, such as various known proprietary operating systems or open-source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The descriptions of the various aspects and/or embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the aspects and/or embodiments, the practical application or technical improvement over technologies found in the marketplace, or to enable others of ordinary skill in the art to understand the aspects and/or embodiments disclosed herein.

### EXPERIMENTAL

The following examples are set forth to provide those of ordinary skill in the art with a complete disclosure of how to make and use the aspects and embodiments of the invention as set forth herein. While efforts have been made to ensure accuracy with respect to variables, experimental error and deviations should be considered.

### Example 1

Process for Introducing Noise into an Input Matrix

A python implementation of the noise algorithm described in paragraph 0044 herein was used with a numpy library to generate the noise matrices shown in FIGS. 2A-2C, which were displayed using the matlibplot library.

### Example 2

### Process for Generating Persistent Homology Barcodes

Applying the noise introduction process of Example 1, real+noise and random permuted data matrices were generated from a real data matrix. Python pandas and numpy libraries were used to generate the filtration matrices using Jaccard similarity and min(com1/sum(1)) computations. Persistent homology barcodes were generated for H0 and H1 dimensions as shown in FIGS. 3A-3C and H0, H1, and H2 dimensions as shown in FIGS. 4A-4B using predefined packages, including matilda, javaplex, and giotto. The input and random data matrices of FIGS. 4A-4B are subsets of elements from the input and random matrices of FIGS. 3A-3C.

We claim:

1. A computer-implemented method for analyzing nestedness of microbes in a microbial community comprising:
generating an input matrix with microbe data comprising 1s and 0s for samples taken from a microbial community, wherein 1 represents the presence of a microbe in the samples and 0 represents the absence of microbes in the samples;
generating a filtration matrix that identifies similarities between microbes in the samples; and
generating at least one persistent homology barcode for the filtration matrix, wherein the at least one persistent homology barcode has a shape and length that provides information on the nestedness of the microbes in the microbial community.

2. The computer-implemented method of claim 1, wherein the number of 1s in the matrix represents abundance of microbes in the samples.

3. The computer-implemented method of claim 1, wherein the filtration matrix is generated with a mathematical function selected from the group consisting of Jaccard similarity, overlap coefficient, or a min(com1/sum(1)) computation.

4. The computer-implemented method of claim 1, wherein the persistent homology is generated with a mathematical function selected from the group consisting of simplicial complex, a cubical complex, an alpha complex, a C̆ech complex, or a Vietris-Rips complex.

5. The computer-implemented method of claim 1, wherein a p-value of the nestedness in the microbial community is calculated by comparing the barcodes generated from the input matrix against barcodes generated from the input matrix with randomly permutated microbe data.

6. The computer-implemented method of claim 5, wherein the microbe data is randomly permutated by introducing noise into the samples, wherein the input matrix and the randomly permutated input matrix have the same number of 1s and 0s.

7. A computer-implemented method for analyzing nestedness in a microbial community comprising:
generating an input matrix with 1s and 0s for one or more samples from the microbial community, wherein 1 represents the presence of a microbe in the one or more samples and 0 represents the absence of microbes in the one or more samples;
computing a filtration matrix for the one or more samples;
computing a C̆ech complex persistent homology for the filtration matrix in at least two dimensions; and
displaying the persistent homology of the filtration matrix with at least one persistent homology barcode, wherein the at least one persistent homology barcode has a shape and length that provides information on the nestedness of the microbes in the microbial community.

8. The computer-implemented method of claim 7, wherein the number of 1s in the matrix represents abundance of microbes in the one or more samples.

9. The computer-implemented method of claim 7, wherein the filtration matrix is generated with a mathematical function selected from the group consisting of Jaccard similarity, overlap coefficient, or a min(com1/sum(1)) computation.

10. The computer-implemented method of claim 7, wherein a p-value of the nestedness in the microbial community is calculated by comparing the persistent homology barcode for the input matrix against a persistent homology barcode for a completely randomized input matrix.

11. The computer-implemented method of claim 10, wherein the input matrix is randomly permutated by introducing noise into the one or more samples, wherein the input matrix and the randomly permutated input matrix have the same number of 1s and 0s.

12. A computer-implemented method for analyzing nestedness in a microbial community comprising:
generating an input matrix with 1s and 0s for one or more samples from the microbial community, wherein 1 represents the presence of a microbe in the one or more samples and 0 represents the absence of microbes in the one or more samples;
computing a Jaccard similarity filtration matrix for the one or more samples;
computing a C̆ech complex persistent homology for the filtration matrix in at least two dimensions; and
displaying the persistent homology of the filtration matrix with at least one persistent homology barcode, wherein the at least one persistent homology barcode has a shape and length that provides information on the nestedness of the microbes in the microbial community.

13. The computer-implemented method of claim 12, wherein the number of 1s in the matrix represents abundance of microbes in the one or more samples.

14. The computer-implemented method of claim 12, wherein a p-value of the nestedness in the microbial community is calculated by comparing the persistent homology barcode for the input matrix against a persistent homology barcode for a completely randomized input matrix.

15. The computer-implemented method of claim 14, wherein the input matrix is randomly permutated by introducing noise into the one or more samples, wherein the input matrix and the randomly permutated input matrix have the same number of 1s and 0s.

16. A computer-implemented method for analyzing nestedness in a microbial community comprising:
generating an input matrix with 1s and 0s for one or more samples from the microbial community, wherein 1 represents the presence of a microbe in the one or more samples and 0 represents the absence of microbes in the one or more samples;
computing a filtration matrix with a min(com1/sum(1)) computation for the one or more samples;
computing a C̆ech complex persistent homology for the filtration matrix in at least two dimensions; and
displaying the persistent homology of the filtration matrix with at least one persistent homology barcode, wherein the at least one persistent homology barcode has a shape and length that provides information on the nestedness of the microbes in the microbial community.

17. The computer-implemented method of claim 16, wherein the number of 1s in the matrix represents abundance of microbes in the one or more samples.

18. The computer-implemented method of claim 16, wherein a p-value of the nestedness in the microbial community is calculated by comparing the persistent homology barcode for the input matrix against a persistent homology barcode for a completely randomized input matrix.

19. The computer-implemented method of claim 18, wherein the input matrix is randomly permutated by introducing noise into the one or more samples, wherein the input matrix and the randomly permutated input matrix have the same number of 1s and 0s.

20. A computer program product for analyzing nestedness in a microbial community comprising:
program instructions on one or more computer readable storage media for generating an input matrix with 1s and 0s for one or more samples from the microbial community, wherein 1 represents the presence of a microbe in the one or more samples and 0 represents the absence of microbes in the one or more samples;
program instructions on one or more computer readable storage media for computing a filtration matrix for the one or more samples;
program instructions on one or more computer readable storage media for computing persistent homology for the filtration matrix in at least two dimensions; and
program instructions on one or more computer readable storage media for displaying the persistent homology of the filtration matrix with at least one persistent homology barcode, wherein the at least one persistent homology barcode has a shape and length that provides information on the nestedness of the microbes in the microbial community.

21. The computer program product of claim 20, wherein the number of 1s in the matrix represents abundance of microbes in the one or more samples.

22. The computer program product of claim 20, wherein the filtration matrix is generated with a mathematical function selected from the group consisting of Jaccard similarity, overlap coefficient, or a min(com1/sum(1)) computation.

23. The computer program product of claim 20, wherein the persistent homology is generated with a mathematical function selected from the group consisting of simplicial complex, a cubical complex, an alpha complex, a C̆ech complex, or a Vietris-Rips complex.

24. The computer-implemented method of claim 20, wherein a p-value of the nestedness in the microbial community is calculated by comparing the persistent homology barcode for the input matrix against a persistent homology barcode for a completely randomized input matrix.

25. The computer program product of claim 24, wherein the input matrix is randomly permutated by introducing noise into the one or more samples, wherein the input matrix and the randomly permutated input matrix have the same number of 1s and 0s.
